(19) United States
(12) Patent Application Publication
(10) Pub. No.: US 2013/0316024 A1
(43) Pub. Date: Nov. 28, 2013

(54) COSMETIC KIT AND USE FOR IMPROVING THE APPEARANCE OF THE SKIN

(71) Applicants: Gabriel Gorescu, Santo Andre (BR); Thomas Canova, Jardim Bela Vista (BR); Tarcis Cordeiro Bastos, Villa Mariana (BR); Karla Laguens, Jardim Paulista (BR)

(72) Inventors: Gabriel Gorescu, Santo Andre (BR); Thomas Canova, Jardim Bela Vista (BR); Tarcis Cordeiro Bastos, Villa Mariana (BR); Karla Laguens, Jardim Paulista (BR)

(73) Assignee: RHODIA POLIAMIDA E ESPECIALIDADES LTDA, Sao Paulo (BR)

(21) Appl. No.: 13/989,386

(22) PCT Filed: Nov. 18, 2011
(86) PCT No.: PCT/IB2011/002746

§ 371 Date: Aug. 9, 2013

(30) Foreign Application Priority Data
Nov. 23, 2010 (FR) FR1059654

(51) Int. Cl. A61K 8/02

(52) U.S. Cl. CPC A61K 8/02
USPC 424/728; 514/161; 424/752; 424/725; 514/263.34; 424/729

(57) ABSTRACT

A cosmetic kit and the use thereof for improving the appearance of the skin is described. Further described, is the prevention and/or controlling of cellulite or the orange peel syndrome and/or for slimming down the figure, through the increase of the elasticity and firmness of the skin by stepping up collagen synthesis, for increasing blood microcirculation and for improving thermoregulation of the skin.

The subject of the present invention is a cosmetic kit and the use thereof for improving the appearance of the skin, in particular for preventing and/or combating cellulite or the orange peel syndrome and/or for slimming down the figure, for increasing the elasticity of the skin, for stepping up collagen synthesis, for increasing blood microcirculation and for improving thermoregulation of the skin.

Human skin consists of three superimposed tissues: the epidermis, which is the outermost tissue, the dermis, and the hypodermis, which is the deepest tissue.

Natural human epidermis is composed mainly of three cell types, which are the keratinocytes, very predominant, the melanocytes and the Langerhans cells. Each of these cell types contributes by its own functions to the essential role played in the body by the skin.

The dermis provides the epidermis with a solid support. It is also its feeder element since it contains a vascularization, which the epidermis does not. It consists mainly of fibroblasts and of an extracellular matrix which is itself composed of various extra-cellular proteins, among which are, in particular, collagen fibers, elastin and various glycoproteins.

The hypodermis, which invaginates into the dermis and is attached to the overlying dermis by collagen fibers and elastin fibers, essentially consists of a type of cells which specialize in fat accumulation and storage, the adipocytes. It is the energy reservoir of the body.

Adipocytes are mature cells which result from a process of differentiation of fibroblasts into pre-adipocytes and adipocytes; this process is called adipogenesis and/or adipocyte differentiation.

Chubbiness and/or excess weight are linked to the dysfunction of these adipocytes, which contain variable amounts of fats stored in the form of triglycerides. These triglycerides are synthesized in vivo by the adipocytes themselves, according to enzymatic-type reactions (lipogenesis), from the free fatty acids contained in the blood in the form of lipoproteins (via a lipoprotein lipase) and from glucose provided in particular by means of certain foods (via an acetyl-CoA-carboxylase).

In parallel, the triglycerides thus formed, and then stored, in the adipocyte cells can also degrade (lipolysis), still under the action of specific enzymes, triglyceride lipases, contained in these same cells, and which are capable of being activated by cyclic AMP, itself regulated by adenyl cyclase and capable of being hydrolyzed to 5′AMP by phosphodiesterase.

If, for various reasons (excessively rich or unbalanced food, inactivity, variation in metabolism, aging and the like), a substantial imbalance becomes established in the body between lipogenesis and lipolysis, i.e., more specifically, if the amounts of fats formed by lipogenesis become appreciably and constantly higher than those which are removed by lipolysis, an accumulation of triglycerides then takes place in the adipocytes, which, if it becomes excessive, may gradually be reflected by deformation of the skin caused by thickening of the hypodermis which contains the adipocytes.

Likewise, an increase is noted in the subcutaneous pool of adipocytes through the recruitment of fibroblasts which will initiate a process of maturing into pre-adipocytes and adipocytes. The surface of the skin becomes irregular (“comprising cellulite” or “orange peel syndrome”) and of more or less flaccid or gelatinous consistency, finally giving the figure an unsightly general appearance, which may vary between simple local excess weight (lipodysmorphia), passing through a certain level of plumpness, and finally to genuine obesity.

As it happens, taking into account in particular the deep physical and esthetic, and sometimes psychological, discomfort experienced by individuals suffering therefrom, in particular in the case of women, adiposity is nowadays a condition that is less and less well tolerated or accepted.

Among the existing solutions for reducing cellulite, and thus improving the esthetics and the well-being of the individual, new cosmetic active agents are regularly being discovered.

Active agents for combating thickening of the hypodermis, which are molecules of synthetic or natural origin, have biological mechanisms. Indeed, these molecules target mature adipocytes, by intervening in fatty acid metabolism which is one of the preferred targets in the control of this lipid overload in adipocytes. This metabolism can be modulated:
either by blocking glucose transport inside the adipocyte, which results in a decrease in fatty acids entering the adipocyte;
or by inhibition of lipoprotein lipase;
or by activation of triglyceride lipase (or hormone-sensitive lipase), generally by stimulating cyclic AMP, generally by activation of adenyl cyclase, or by causing the accumulation thereof by inhibiting phosphodiesterase.

Other biological approaches have been explored for acting on the mechanism of lipogenesis and/or lipolysis. It has thus been proposed to use neuropeptide Y (NPY) receptor antagonists, neuropeptide Y being a neuromediator involved in a certain number of physiological processes and for which it has been possible to demonstrate its involvement in the regulation of lipolysis (P. Valet, J. Clin. Invest., 1990, 85, 291-295). a2 receptor antagonists or else 83-adrenergic receptor agonists can also be used. It is also possible to use regulation by cytosoluble factors, by using activators of interleukin 11 (IL11) production, by skin epidermal cells and/or sebaceous gland cells.

All these active agents are generally formulated in creams or gels, in which other constituents (other active agents or excipients) are present.

The problems associated with this type of product are that the results are not significantly observed by users, who rapidly lose interest. Furthermore, topical lotions can leave marks which can stain clothing worn after having applied the formulation or else leave a tacky skin sensation.

Furthermore, it has been found, as can be seen in particular in WO 2009/077834, that additives having properties of emission and/or absorption in the infrared region also interact with the skin. These additives can be formed in polymeric compositions, in particular polyamide-based compositions, which are then spun so as to constitute “active” fibers usable, for example, to design textile articles for reducing cellulite. In this case, these textile articles operate physically/thermally, i.e. a thermoregulation occurs at the surface of the skin, by virtue of which the blood microcirculation is activated, and which promotes elimination of cellulite.

Products also exist which combine cosmetic formulae (creams or gels) with fabrics which are either nonactive or active with the same operating principle as the cosmetic formula. Mention may be made of, for example:
nonactive tights/cosmetic cream or gel: the tights do not have an isolated action in terms of improving the cellulite; they merely represent an aid to maintaining the cosmetic formulation on the skin;
aluminum patch/cosmetic cream: the patch assists in maintaining and accelerating the absorption of the cosmetic active agent;
Bermuda shorts covered with bioceramics/cream containing bioceramics: the Bermuda shorts coated with bioceramic and the cosmetic cream containing bioceramics have the same mechanism of action and no additional effect associated with the use of the two products is observed;
fabrics impregnated with microcapsules of cosmetic active agents. In this case, the function of the fabric is to support the active substance, which is generally released because of fabric/skin friction. The drawbacks of this technology are the following:
   release of the additives is too rapid and the active agents disappear after a few washes;
   amount of active agent applied is insufficient to observe an effect since the volume of microcapsules is very small compared with the amount applicable by means of a cosmetic cream;
   active formulation used is identical to that of cosmetic creams and lotions.

There is therefore still a need to provide new “anticellulite” solutions which satisfy the users of such cosmetic products. The need lies in an increase in the effectiveness of the elimination of the unsightly forms caused by the subcutaneous aggregates of fat.

One of the objectives of the present invention is therefore to propose a new, more effective product which does not have the drawbacks mentioned above.

The invention meets this need by proposing a cosmetic kit which makes it possible to significantly improve the appearance of the skin, in particular the roughness and the firmness of the skin which are modified owing to the presence of cellulite.

More specifically, the invention relates to a cosmetic kit comprising:
a topical formulation comprising at least one cosmetic active agent, and
a textile article constituted at least in part by polymer fibers which have a capacity for emission and/or absorption of infrared radiation in the wavelength range located between 2 μm and 20 μm.

The present invention is also directed toward the cosmetic use of the kit of the invention for improving the appearance of the skin, in particular for preventing and/or combating cellulite or the orange peel syndrome and/or for slimming down the figure. In particular, the cosmetic use according to the invention makes it possible to increase the elasticity and the firmness of the skin, to step up collagen synthesis, to increase blood microcirculation and to improve thermoregulation of the skin.

The kit of the invention uses a topical formulation comprising at least one cosmetic active agent.

The cosmetic active agent according to the invention is advantageously an active agent which has a cellulite-reducing effect. In the context of the invention, the expression “cellulite-reducing effect” is intended to mean a compound, the activity of which results in reducing the thickness of the hypodermis and/or preventing thickening thereof, in particular by controlling the renewal and/or the number of adipocytes, via an inhibition and/or a decrease in the conversion of fibroblasts into pre-adipocytes and adipocytes.

Controlling the renewal and/or the number of adipocytes in the hypodermis thus makes it possible to:
reduce the thickness of the hypodermis and/or prevent thickening thereof;
prevent and/or combat cellulite and/or the orange peel appearance, and/or slim down the figure or make it thinner.

The topical formulation of the invention can be in the form in particular of an aqueous, alcoholic or aqueous-alcoholic solution or suspension or an oily suspension or a solution or dispersion of the lotion or serum type, an emulsion of liquid or semi-liquid consistency of the milk type, obtained by dispersion of a fatty phase in an aqueous phase (oil-in-water) or vice versa (water-in-oil), or a triple emulsion (water-in-oil-in-water or oil-in-water-in-oil), or a suspension or emulsion of soft, semi-solid or solid consistency of the cream or gel type, microemulsions, or vesicular dispersions of ionic and/or nonionic type.

These formulations are prepared according to the usual methods.

This formulation may be more or less fluid and have the appearance of a white or colored cream, an ointment, a milk, a lotion, a serum, a paste or a foam. It may optionally be applied to the skin in the form of an aerosol. It may also be in solid form, and for example in the form of a stick. Preferably, the formulation according to the invention will be in the form of a lotion, a serum, a milk, a cream, an oil, a stick, a patch or else a spray or aerosol. When the formulation is an emulsion, the proportion of the fatty phase can range from 2% to 80% by weight and preferably from 5% to 50% by weight relative to the total weight of the formulation. The oils, the waxes, the emulsifiers and the coemulsifiers used in the formulation in emulsion form are chosen from those conventionally used in the cosmetics field. The emulsifier and the coemulsifier are present, in the formulation, in a proportion ranging from 0.1% to 30% by weight and preferably from 0.5% to 20% by weight relative to the total weight of the formulation.

The emulsion may also contain lipid vesicles.

When the formulation is an oily solution or gel, the fatty phase may represent more than 90% of the total weight of the formulation.

In a known manner, the formulation of the invention may also contain the adjuvants which are customary in the cosmetics fields, such as hydrophilic or lipophilic gelling agents, hydrophilic or lipophilic active agents, preservatives, antioxidants, solvents, fragrances, fillers, screening agents, pigments, chelating agents, odor absorbers and colorants.

The amounts of these various adjuvants are those conventionally used in the fields under consideration, and are for example from 0.01% to 20% of the total weight of the formulation. Depending on their nature, these adjuvants can be introduced into the fatty phase, into the aqueous phase, into lipid vesicles and/or into nanoparticles.

As oils or waxes that can be used in the invention, mention may be made of mineral oils (liquid petroleum jelly), vegetable oils (liquid fraction of shea butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers that can be used in the invention, mention may, for example, be made of glyceryl stearate, polysorbate 60 and the mixture of PEG-6/PEG-32/glycol stearate sold under the name Tefose R 63 by the company Gattefosse.

As solvents that can be used in the invention, mention may be made of lower alcohols, in particular ethanol and isopropanol, and propylene glycol.

As hydrophilic gelling agents that can be used in the invention, mention may be made of carboxyvinyl polymers (carbomer), acrylic copolymers such as copolymers of acrylates/alkyl acrylates, polyacrylamides, polysaccharides such as hydroxypropylcellulose, natural gums and clays, and, as lipophilic gelling agents, mention may be made of modified clays such as bentones, metal salts of fatty acids such as aluminum stearates and hydrophobic silica, ethylcellulose and polyethylene.

Among the cosmetic active agents capable of having a cellulite-reducing effect, mention may be made of:
1) phosphodiesterase inhibitors,
2) plant extracts and extracts of marine origin,
3) peptides or proteins,
4) active agents which act on the microcirculation (vasoprotectors or vasodilators),
5) firming active agents and/or anti-glycation active agents (which prevent the binding of sugar to collagen fibers) and/or antioxidant active agents,
6) antilipogenesis agents: interleukin 11 activators.

The phosphodiesterase inhibitors are advantageously chosen from:
xanthine derivatives, such as caffeine and derivatives thereof, in particular the 1-hydroxyalkylxanthines described in document FR-A-2 617 401, caffeine citrate, theophylline and derivatives thereof, theobromine, acefylline, aminophylline, chloroethyltheophylline, diprofylline, diniprophylline, etamiphylline and its derivatives, etofylline and proxyphylline;
combinations containing xanthine derivatives, such as the combination of caffeine and silanol (caffeine methylsilanetriol derivative), and for example the product sold by the company Exsymol under the name cafeisilane C;
compounds of natural origin containing xanthine bases, and in particular caffeine, such as extracts of tea, of coffee, of guarana, of mate, of cola (Cola nitida) and in particular the dry extract of guarana fruit (Paulina sorbilis) containing 8% to 10% of caffeine;
ephedrine and its derivatives which may be found in particular in natural form in plants such as Ma Huang (Ephedra plant).

As plant extracts and extracts of marine origin, which are either active on the receptors to be inhibited, such as J3-2-blockers, NPY-blockers (described in patent EP 838 217), or inhibit the synthesis of LDL or VLDL receptors, or are active in stimulating R receptors and G proteins, leading to the activation of adenyl cyclase, mention may, for example, be made of:
plant extracts:
   Garcinia cambogia,
   extracts of Bupleurum chinensis,
   extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's broom (Ruscus aculeatus L), of meadowsweet (Filipendula ulmaria L), of orthosiphon (Orthosiphon stamincus benth), of birch (Betula alba), of pumpwood and of argan tree,
   extracts of ginkgo biloba,
   extracts of horsetail,
   extracts of escin,
   extracts of cangzhu, extracts of Chrysanthemum indicum, extracts of dioscorea rich in diosgenin or pure diosgenin or hecogenin, and derivatives thereof,
   extracts of plants of the genus Armeniacea, Atractylodis, Platycodon, Sinomenium, Pharbitidis or Flemingia,
   extracts of Coleus, such as C. forskohlii, C. blumei, C. esquirolii, C. scutellaroides, C. xanthantus and C. barbatus, such as the extract of root of Coleus barbatus containing 60% forskolin,
   extracts of Ballota,
   extracts of Guioa, of Davallia, of Terminalia, of Barringtonia, of Trema or of Antirobia,
   extracts of Camellia sinensis leaf;
extracts of marine origin: extracts of algae or of phytoplankton, such as rhodysterol or the extract of Laminaria digitata sold under the name Phycox75 by the company Secma, the alga Skeletonema described in patent FR 2 782 921 or the diatoms described in patent FR 2 774 292.

The peptides or proteins are preferably chosen from:
peptides derived from parathyroid hormone, as described in patents FR 2 788 058 and FR 2 781 231 from Sederma or the peptides described in document FR 2 786 693, or even any other peptide having lipolytic properties,
protamines and derivatives thereof, such as those described in document FR-A-2 758 724.

Among the active agents which act on the microcirculation (vasoprotectors or vasodilators), mention may be made of flavonoids, ruscogenins, natural or synthetic esculosides (including Permethol sold by the company Sochibo), escin extracted from horse chestnut, nicotinates, hesperidin methyl chalcone, ruscus, essential oils of lavender or rosemary, and extracts of Ammi visnaga.

Among the firming active agents and/or anti-glycation active agents (which prevent the binding of sugar to collagen fibers) and/or antioxidant active agents, mention may be made of extracts of Centella asiatica and of Siegesbeckia, silicon, amadorine, ergothioneine and its derivatives, plant extracts of the family Ericaceae, in particular extracts of blueberry (Vaccinium angustifolium), and vitamin C and its derivatives.

Preferably, the cosmetic active agent is chosen from phosphodiesterase inhibitors, active agents which act on the microcirculation (vasoprotectors or vasodilators), firming active agents and/or anti-glycation active agents (which prevent binding of sugar to collagen fibers) and/or antioxidant active agents, antilipogenesis agents, and mixtures thereof.

The amount of cosmetic active agent can vary to a large extent and depends on the nature of the active agent(s) used. Generally, the cosmetic active agent(s) are present in a concentration ranging from 0.0001% to 20%, preferably from 0.001% to 10% and even more preferentially from 0.1% to 5% by weight relative to the total weight of the formulation.

According to one advantageous embodiment, the topical formulations according to the invention contain at least two cosmetic active agents.

The cosmetic active agent can be either (i) encapsulated in a coating, such as microspheres, nanospheres, oleosomes or nanocapsules, or (ii) compartmentalized in a fatty phase containing the main constituents of sebum.

According to a first embodiment, the formulation can be in a form such that the active agent is encapsulated in the core or the wall of a coating, such as microspheres, nanospheres, oleosomes, niosomes or nanocapsules, preferably nanocapsules.

The encapsulation or the absorption of lipophilic active ingredients in particles of submicronic size has been known for several years and is widely used, in particular in the cosmetics and dermatology fields. Indeed, these particles, called nanoparticles, are capable of crossing the superficial layers of the stratum corneum and/or of the follicular ostium and of penetrating into the upper layers of the live epidermis so as to release the active ingredient therein. This penetration into deeper layers broadens the space in which the active ingredients can act and protects them from rapid elimination by simple friction.

The term “nanoparticles” encompasses mainly two different systems: ‘nanospheres’ consisting of a polymer matrix in which the active ingredient is absorbed and/or adsorbed and/or mixed, and also ‘nanocapsules’ which have a core-shell structure, i.e. a structure consisting of a lipid core which is liquid at ambient temperature, formed of or containing the active ingredient in solubilized or pure form, which core is encapsulated in a continuous protective shell that is insoluble in the medium.

The nanocapsules according to the present invention are generally small in size in order to obtain optimum bioavailability of the active compound. Preferentially, the size of these nanocapsules is between 10 nm and 1000 nm and more particularly between 30 nm and 500 nm.

Various types of nanocapsules can be used according to the present invention. By way of example, mention may be made of the nanocapsules described in patent application EP-0 274 961, the nanocapsules provided with a lamellar coating which are described in application EP-0 780 115, the nanocapsules of which the water-insoluble continuous polymeric shell consists of polyesters, as described in applications EP-1 025 901, FR-2 787 730 and EP-1 034 839, or else the biodegradable nanocapsules described in patent application FR-2 659 554, or the nonbiodegradable nanocapsules described in patent application WO 93/05753.

The nanocapsules made of biodegradable polymers penetrate into the skin and degrade in the epidermis under the action of the enzymes which are present therein, whereas the nanocapsules made of nonbiodegradable polymers penetrate only into the superficial layers of the stratum corneum and are eliminated naturally during renewal of the skin.

Use may also be made of systems which deliver the active product specifically into the pilosebaceous unit, such as microspheres of natural or synthetic polymers or of fatty substances with a melting point above 50° C., charged with at least one active product, at least 80% by weight of these microspheres having a diameter between 3 pm and pm, described in application EP 0 375 520; or vehicles consisting of porous particles with a size of between 10 pm and 100 pm for controlled release of the active ingredients, described in patent U.S. Pat. No. 4,690,825.

The kit of the invention uses a textile article constituted at least in part by polymer fibers which have a capacity for emission and/or absorption of infrared radiation in the wavelength range located between 2 μm and 20 μm.

The polymer fibers according to the invention preferably have a number of infrared radiation absorption peaks greater than 10 in the following ten frequency ranges: 3.00 +/−0.30 μm, 6.20+/−0.50 μm, 8.00+/−0.25 μm, 8.50+/−0.25 μm, 9.00+/−0.25 μm, 9.50+/−0.25 μm, 10.00+/−0.25 μm, 10.50+/−0.25 μm, 11.00+/−0.25 μm, 14.60+/−2.10 μm, at least 1 peak being present in at least 7 of these ten frequency ranges.

The infrared radiation absorption spectrum can be determined by any method known to those skilled in the art. One possible method is the use of a Bruker Equinox 55 instrument, with a resolution of 4 cm⁻¹. In this case, the spectrum obtained is in ATR (attenuated total reflectance) form, using a ZnSe crystal.

The polymer can be chosen from the group comprising polyesters, polyolefins, cellulose-ester-based polymers such as cellulose acetate, cellulose propionate, rayon, viscose and polymers of the same family, acrylic polymers and copolymers, polyamides, polyhexamethylene adipamide (PA66) or polycaproamide (PA6), or copolymers thereof in any proportions, or else blends between any polymers mentioned above.

According to one preferential embodiment, the polymer of which the fiber is composed is based on polyamide, chosen from polyamide 6, polyamide 66 and copolymers of polyamide 6/polyamide 66 in any proportions.

The polymer fibers according to the invention comprise, in the polymer matrix, inorganic fillers having properties of absorption and/or emission in the 2-20 μm far infrared region.

The inorganic fillers are chosen from oxides, sulfates, carbonates, phosphates and silicates.

Preferably, the oxide is chosen from titanium dioxide, silicon dioxide and magnesium oxide.

The sulfate can be chosen from alkali metal sulfates, preferably from barium sulfate, calcium sulfate and strontium sulfate.

The carbonate is advantageously chosen from calcium carbonate or sodium carbonate.

Preferably, the silicate is chosen from aluminosilicates, preferably from actinolite, tourmaline, serpentine and kaolin.

The phosphate can be chosen from zirconium phosphates, apatite, or mixtures thereof.

According to one advantageous embodiment of the invention, the inorganic fillers are of at least one type chosen from oxides, sulfates, carbonates, phosphates and silicates. Preferably, the inorganic fillers are of two types chosen from the following types: oxides, sulfates, carbonates, phosphates and silicates. Even more advantageously, the inorganic fillers are of three types chosen from the following types: oxides, sulfates and silicates.

Particularly advantageously, the fiber comprises three inorganic fillers.

Preferably, at least two inorganic fillers chosen from titanium dioxide, an alkali metal sulfate and an aluminosilicate. Preferably, at least two inorganic fillers chosen from titanium dioxide, barium sulfate and tourmaline are present in the fiber.

The combination of the three inorganic fillers is preferably the titanium dioxide/alkali metal sulfate/aluminosilicate combination, preferably the titanium dioxide/barium sulfate/tourmaline combination.

In this case, the proportion by weight of the three inorganic fillers is between 80:10:10 and 10:30:60, and more specifically in proportions of 50:25:25.

According to one embodiment of the invention, the proportion by weight of the combination of inorganic fillers relative to the total weight of the polymeric composition is greater than 1.0%, preferably greater than or equal to 1.5% and even more preferentially greater than or equal to 2.5%.

Preferably, the proportion by weight of the combination of inorganic fillers relative to the total weight of the polymeric composition is less than 9%, preferably less than 6% and advantageously less than 4.5%.

The inorganic fillers according to the invention advantageously have an average particle size of less than 2 μm.

The inorganic fillers of the polymeric composition advantageously have a particle size of less than 1.0 μm and preferably a particle size of less than 0.5 μm.

The inorganic fillers are incorporated during the polymer synthesis phase, or by direct mixing with the polymer during the filament spinning phase, or else by means of a concentrate of particles in the form of a masterbatch, it being possible for the latter to be subsequently diluted to predetermined concentrations in the polymer mass during the spinning phase.

The process for obtaining such fibers according to the invention can consist in preparing a suspension of the inorganic fillers, for instance an aluminosilicate, titanium dioxide and an alkali metal sulfate, stabilized by surfactants. The suspension is then added to the synthesis of the polyamide. An alternative is to introduce a part of the inorganic fillers, previously made into the form of a masterbatch, into the molten polymer at the time of spinning. The polyamide obtained is cooled, cut and remelted before passing through an extruder so as to form the fiber.

The polyamide fibers according to the invention contain the inorganic fillers uniformly dispersed in the polymer matrix.

In the case of fibers obtained by melt spinning, the inorganic fillers can be introduced into the molten polymer by means of a mixing device, for example upstream of a spinning device. Continuous multifilament yarns, monofilaments, short and long fibers, or mixtures thereof, can be obtained by spinning the additive-containing polymer composition. The term “fiber” will be used to refer to all yarns, fibers and filaments that can be obtained by spinning. The fibers obtained from the polymeric compositions presented in the present invention can be subjected to any of the textile treatments known to those skilled in the art, such as extrusion, drawing, texturing, dyeing, finishing, etc.

According to one particularly preferred embodiment of the invention, at least a part of the polymer fibers used in the textile article are fibers sold under the names “FIR emitter yarns” or Emana® by the company Rhodia. They are polyamide 66 fibers comprising three inorganic fillers: titanium dioxide, an alkali metal sulfate and an aluminosilicate.

The textile articles can be obtained from a single type of fiber, or from a mixture of fibers of different types.

The term “textile articles” is intended to mean in particular fabrics, knits and nonwovens.

The textile articles are manufactured by known techniques using the polyamide fibers as starting material.

The interaction between the textile article and the skin creates a thermoregulation and promotes the blood microcirculation.

A reduction in cellulite results from such a stimulation. Furthermore, an improvement in muscle recovery and skin elasticity is observed.

According to one particularly advantageous embodiment, the textile article is in the form of a piece of clothing, preferably Bermuda shorts, tights, a pair of trousers or any other textile article in direct contact with the skin.

The use of a textile article as defined above, in combination with the topical formulation comprising a cosmetic active agent, allows better absorption and better transport of the active agents of the topical formulation, thus generating better elimination of the compounds targeted by the cosmetic active agents.

The invention therefore also relates to the cosmetic use of the cosmetic kit as defined above, for improving the appearance of the skin, in particular for preventing and/or combating cellulite or the orange peel syndrome and/or for slimming down the figure, in particular by increasing the elasticity and the firmness of the skin.

More specifically, the topical formulation and the textile article are used successively.

In particular, the use of the cosmetic kit comprises the topical application to the skin of the topical formulation comprising at least one cosmetic active agent, followed by the application, on the area of the skin where the topical formulation was applied, of the textile article constituted at least in part by polymer fibers which have a capacity for emission and/or absorption of infrared radiation in the wavelength range located between 2 μm and 20 μm.

Preferably, the application of the textile article having a capacity for emission and/or absorption of infrared radiation in the wavelength range located between 2 μm and 20 μm consists in wearing the textile article, in particular in the form of a piece of clothing, for example Bermuda shorts, tights or a pair of trousers.

The kit according to the invention can be used locally on the areas of the body marked by a thickening of the hypodermis.

In particular, the topical formulation comprising at least one cosmetic active agent will be applied to the areas of the body to be slimmed down, in particular the hips, the buttocks, the thighs, the stomach and the waistline. A piece of clothing made at least in part from polymer fibers which have a capacity for emission and/or absorption of infrared radiation in the wavelength range located between 2 μm and 20 μm will then be worn.

It is recommended that the piece of clothing be worn preferentially for a minimum of 6 hours a day. Preferably, the application of the topical formulation followed by the wearing of the textile article will be carried out daily, for example once or twice a day, for a treatment period of several weeks, generally from 10 days to 8 weeks and preferably from 2 to 8 weeks.

For better efficiency, the piece of clothing should be worn a few minutes after the application of the topical formulation, as soon as the skin feels dry.

One of the major advantages of the present invention lies in the possibility of being able to perform, whenever necessary or desirable, very localized and selective “mild” treatments by virtue of the topical mode of application.

The treatment may therefore be renewed periodically, according to the thickness and/or the thickening of the hypodermis and/or to the cellulite and/or to the orange peel syndrome of the individual to be treated. The cosmetic use according to the invention also makes it possible to increase the elasticity and firmness of the skin, to step up collagen synthesis, to increase blood microcirculation and to improve thermoregulation of the skin.

Surprisingly, the combination of the topical formulation and of the textile article makes it possible to obtain greater effectiveness in the reduction of cellulite.

The present invention also has the following advantages:
high resistance to washing/cleaning of the inorganic fillers of the textile article by virtue of the incorporation of these fillers into the polymeric matrix,
the infrared radiation emitting/absorbing textile article amplifies the effect of the topical formula, in particular by thermoregulation of the skin, and thus improves the absorption of the cosmetic active agents,
the infrared radiation emitting/absorbing textile article promotes the transport and the permeability of the topical formulation by virtue of the increase in blood microcirculation of the area treated,
the combination of these two totally different and complementary operating mechanisms makes it possible to obtain a significant improvement in the appearance of the skin.

### DESCRIPTION OF THE FIGURE

FIG. 1 represents a diagram of a graph of the measurement of mechanical deformation of the skin as a function of time according to the cutometry method.

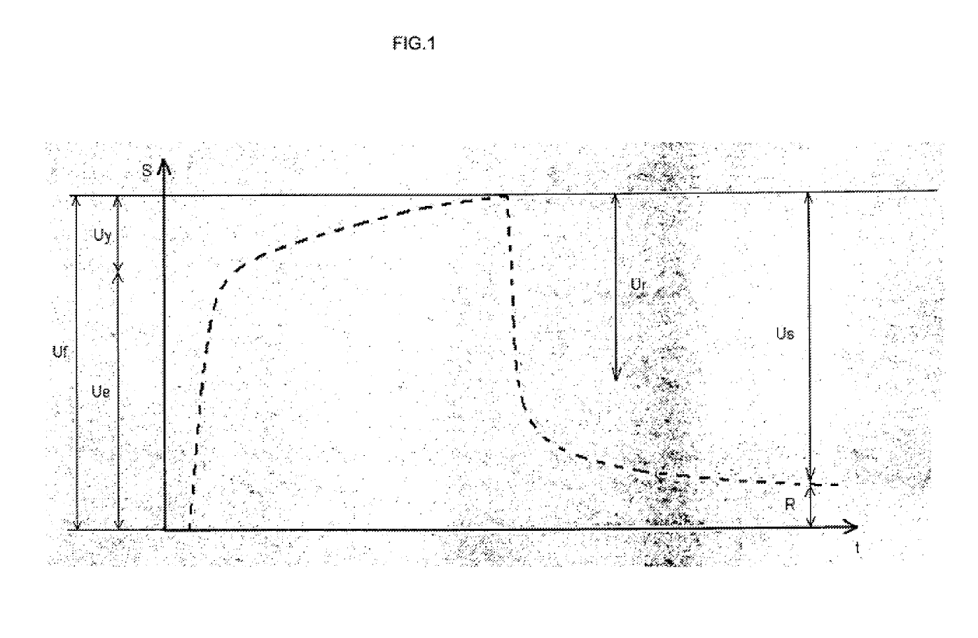

FIG. 1 shows the measurement of this deformation (S) during suction which results in the determination of the following parameters:
maximal extensibility: Uf (final deformation);
immediate extensibility: Ue (elasticity);
viscoelastic extensibility: Uy (plasticity);
immediate elastic recovery: Ur;
elastic deformations ratio (firmness F): Ur/Ue;
elastic recovery rate: Ur/Uf;
viscoelasticity rate: Uy/Ue.

This figure is explained in greater detail in the examples which follow.

Exemplary embodiments of the invention are given hereinafter. These examples are given by way of illustration and are not limiting in nature.

### EXAMPLES

### Tests

Six topical formulation+textile article tests were carried out in order to demonstrate the synergistic effect linked to the combined use of these two elements in the treatment of cellulite.

a) Bermuda shorts made of Emana® infrared-emitting yarns+topical formulation A

b) Bermuda shorts made of Emana® infrared-emitting yarns+topical formulation B

c) Bermuda shorts made of Emana® infrared-emitting yarns+topical formulation C

d) Bermuda shorts made of Emana® infrared-emitting yarns+topical formulation D

e) Bermuda shorts made of Emana® infrared-emitting yarns+topical formulation E

f) Bermuda shorts made of Emana® infrared-emitting yarns+topical formulation F

g) Bermuda shorts made of Emana® infrared-emitting yarns+topical formulation G

h) Fabric made of Emana® infrared-emitting yarns+topical formulation H with:

Topical formulation A: gel comprising 1.5% by weight of caffeine, 0.3% by weight of salicylic acid and 0.3% by weight of retinol.

Topical formulation B: emulsion comprising 2.0% by weight of caffeine, 0.3% by weight of ginkgo biloba and 0.3% by weight of retinol.

Topical formulation C: emulsion comprising 1.5% by weight of caffeine, 0.3% by weight of salicylic acid and 0.3% by weight of extract of butcher's broom (Ruscus aculeatus).

Topical formulation D (commercial formulation Roc—Retinol® Anti-Cellulite): retinol, caffeine, forskolin; tetrahydroxypropyl ethylenediamine (THPE) and carnitine.

Topical formulation E (commercial formulation Biotherm®—Celluli Laser®): complex of fibrins, plankton, caffeine, ginseng, ginkgo biloba, kola nut.

Topical formulation F (commercial formulation Vichy®—Lipocure Serum®): water, alcohol, cyclohexasiloxane, propylene glycol, glycerol, caffeine, dimethicone peg-7 phosphate, triethanolamine, caprylic/capric peg-6, lycerides, carbomer, manganese gluconate, salicylic acid, escin, ginkgo biloba, rutinyl disodium disulfate, dioscorea opposita, xanthan gum, acrylates/C10-30 alkyl acrylate crosspolymer.

Topical formulation G (commercial formulation L'Oreal®—Perfect Slim Lifting Pro®): water, alcohol, propylene glycol, glycerol, caffeine, dimethicone, salicylic acid, silica, mint extract, ginkgo biloba, extract of Ruscus aculeatus, fragrance, extract of wild yam, limonene, linalool.

Topical formulation H (commercial formulation Procter & Gamble®—Olay®): water, glycerol, niacinamide, isohexadecane, dimethicone, isopropyl isostearate, polyacrylamide, sorbitan stearate, cetyl ethanol, sucrose polycottonseedate, tocopheryl acetate, C13-14 isoparaffin, panthenol, benzyl alcohol, titanium dioxide, stearyl ethanol, dimethiconol, ethylparaben, PEG-100 stearate, fragrance, laureth-7, propylparaben, stearic acid, disodium EDTA, sodium ascorbyl phosphate, bht, zinc oxide, citric acid, extract of Camellia sinensis leaf, methylparaben, CI 19140, poly(ammonium acrylate), CI 16035.

### Results

Roughness of the skin:

The reduction in roughness of the skin was measured with a standardized method which consists in analyzing the surface of the skin with a Nikon D70S 3D relief photographic apparatus. The images taken are analyzed using specialized software (Scion software for Windows) which translates the relief into roughness. The roughness is the measurement of the mean variation (standard deviation) of the peaks and valleys of the image.

The standard treatments against cellulite, based on topical formulations (topical formulations A to G) alone or using only a textile article which emits/absorbs in the 2-20 μm wavelength range, show a reduction in the roughness of the skin of 8% to 12%.

The results obtained using the Bermuda shorts made of Emana® infrared-emitting yarns+anti-cellulite formulation (topical formulations A to G) combination for 30 days (6 hours a day) showed an additional reduction of at least 2% of the roughness of the skin compared with the isolated effect of each of the standard methods.

Firmness of the skin:

The increase in firmness of the skin was also measured with a standardized method which consists in analyzing the cutometry: i.e. the measurement of the curve of mechanical deformation of the skin according to FIG. 1.

The principle of this technique consists in creating a reduced pressure at the surface of the skin and in measuring the induced vertical movement of the skin. This technique is therefore based on a suction method. A reduced pressure is created in the probe in order to suction the skin.

The cutometer measures the vertical movement of the surface of the skin as a function of time and of the reduced pressure exerted in the probe. It is thus possible to determine the stiffness of the skin and its ability to return to its initial state.

The measurement of this deformation (S) during the suctioning results in the determination of the following parameters (see FIG. 1):
maximal extensibility: Uf (final deformation);
immediate extensibility: Ue (elasticity);
viscoelastic extensibility: Uy (plasticity);
immediate elastic recovery: Ur;
elastic deformations ratio (firmness F): Ur/Ue;
elastic recovery rate: Ur/Uf;
viscoelasticity rate: Uy/Ue.

The value “F” was measured at time zero (“F₀”) and after 30 days (“F₃₀”), while using the fabric h) for 8 hours.

F(%)=(F₃₀−F₀)/F₀

The results obtained are reported in table 1 below.

TABLE 1

| | | Corrected firmness |
|---|---|---|
| Product | Firmness (F %) | (F′ = F − Fplacebo %) |
| None (placebo) | −1.1% | 0 |
| Fabric made of infrared | 3.2% | 4.3% |
| emitting yarns, alone | | |
| Topical formulation H alone | 8.6% | 9.7% |
| Combination h) fabric made of | 15.4% | 16.5% |
| infrared emitting yarns + | | |
| topical formulation H | | |

The results obtained using the fabric made of infrared emitting yarns+cosmetic formulation H combination for 30 days (8 hours per day) demonstrated a synergistic effect with an additional increase of 6.8% in the firmness of the skin (F%) compared with the best isolated effect of each of the products (i.e. with the formulation alone), or in other words a gain of +2.5% over the sum of the increases obtained with the isolated products (9.70+4.30=14%, whereas the combination reaches 16.5%).

### Conclusions

The clinical studies show that there is a synergistic effect linked to the use of the composition of the invention which comprises both the topical formulation comprising at least one cosmetic active agent and the Rhodia technology of infrared emitting yarns, for treatment of the orange peel appearance and reduction of the cellulite appearance.

1. A cosmetic kit comprising:
a topical formulation comprising at least one cosmetic active agent, and
a textile article constituted at least in part by polymer fibers which have a capacity for emission and/or absorption of infrared radiation in a wavelength range between 2 μm and 20 μm.

2. The cosmetic kit as defined by claim 1, wherein the topical formulation is an aqueous, alcoholic or aqueous-alcoholic solution or suspension or an oily suspension or a solution or dispersion of a lotion or a serum, an emulsion of liquid or semi-liquid consistency of a milk, obtained by dispersion of a fatty phase in an aqueous phase: oil-in-water or vice versa: water-in-oil, or a triple emulsion: water-in-oil-in-water or oil-in-water-in-oil, or a suspension or emulsion of soft, semi-solid or solid consistency of a cream or a gel, microemulsions, or ionic and/or nonionic vesicular dispersions.

3. The kit as defined by claim 1, wherein the cosmetic active agent is an active agent which has a cellulite-reducing effect.

4. The kit as defined by claim 1, wherein the cosmetic active agent is selected from the group consisting of:
1) phosphodiesterase inhibitors,
2) plant extracts and extracts of marine origin,
3) peptides or proteins,
4) active agents which act on the microcirculation,
5) firming active agents and/or anti-glycation active agents and/or antioxidant active agents, and
6) antilipogenesis agents: interleukin 11 activators.

5. The kit as defined by claim 1, wherein the polymer of which the fibers are composed is selected from the group consisting of polyesters, polyolefins, cellulose-ester-based polymers, acrylic polymers and copolymers, polyamides, copolymers thereof, and blends thereof.

6. The kit as defined by claim 1, wherein the polymer of which the fibers are composed is based on polyamide, selected from the group consisting of polyamide 6, polyamide 66 and copolymers of polyamide 6/polyamide 66 in any proportions.

7. The kit as defined by claim 1, wherein the polymer fibers comprise, in the polymer matrix, inorganic fillers which have properties of absorption and/or emission in the 2-20 μm far infrared region.

8. The kit as defined by claim 7, wherein the inorganic fillers are uniformly dispersed in the polymer matrix.

9. The kit as defined by claim 7, wherein the inorganic fillers are selected from the group consisting of oxides, sulfates, carbonates, phosphates and silicates.

10. The kit as defined by claim 7, wherein the inorganic fillers are of three types selected from the group consisting of: oxides, sulfates and silicates.

11. The kit as defined by claim 7, wherein the three inorganic fillers are a titanium dioxide/alkali metal sulfate/alumino-silicate combination.

12. A method of improving the appearance of skin, the method comprising using the topical formulation and the textile article in the kit as defined by claim 1, to improve the appearance of the skin.

13. The method as defined by claim 12, wherein the topical formulation and the textile article are used successively.

14. The method as defined by claim 13, the method further comprising topically applying the topical formulation to the skin and then applying the textile article to an area of the skin where the topical formulation was applied.

15. The method as defined by claim 14, wherein the application of the textile article consists of wearing the textile article as a piece of clothing.
